(19) United States
(12) Patent Application Publication
(10) Pub. No.: US 2012/0185045 A1
(43) Pub. Date: Jul. 19, 2012

(54) ARTICULATING SPINAL IMPLANT INSERTION INSTRUMENT

(75) Inventors: ERIC W. MORRIS, MINNEAPOLIS, MN (US); MARK J. KROLL, ST. PAUL, MN (US); CARSTEN BASTEIN, KIEL (DE)

(73) Assignee: ZIMMER SPINE, INC., MINNEAPOLIS, MN (US)

(21) Appl. No.: 13/006,817

(22) Filed: Jan. 14, 2011

(51) Int. Cl. A61F 2/44; A61B 17/56

(52) U.S. Cl. 623/17.11; 606/86 A

(57) ABSTRACT

An actuatable insertion instrument for inserting a spinal implant in an intervertebral disc space between adjacent vertebrae. The actuatable insertion instrument includes an indicator pivotably coupled to the elongate shaft of the actuatable instrument for providing visual indication of an angular orientation of a spinal implant removably coupled to the insertion instrument. The insertion instrument also includes a knob which may be rotated to adjust the angular orientation of the spinal implant, and a button mounted to the handle which may be actuated between a first position which allows rotation of the knob to articulate the spinal implant, and a second position which prevents rotation of the knob.

### TECHNICAL FIELD

The disclosure is directed to instruments, devices and techniques for use in interbody fusion procedures. More particularly, the disclosure is directed to an actuatable spinal implant insertion instrument and associated techniques for implanting a spinal implant in an intervertebral disc space.

### BACKGROUND

The spinal column of a patient includes a plurality of vertebrae linked to one another by facet joints and an intervertebral disc located between adjacent vertebrae. The facet joints and intervertebral disc allow one vertebra to move relative to an adjacent vertebra, providing the spinal column a range of motion. Diseased, degenerated, damaged, or otherwise impaired intervertebral discs may cause the patient to experience pain or discomfort and/or loss of motion, thus prompting surgery to alleviate the pain and/or restore motion of the spinal column.

In some circumstances in which an intervertebral disc becomes damaged or diseased, upon removal of the intervertebral disc, or a portion thereof, an intervertebral spinal implant may be inserted into the disc space between the vertebral bodies of the adjacent vertebrae to maintain the distance between the vertebral bodies and to enable stabilization of the adjacent vertebrae through bone fusion.

Access to a damaged disc space with an intervertebral implant may be accomplished from several approaches to the spinal column. For example, in some instances an anterior approach may be used through the patient's abdomen. In other instances, a posterior approach or a lateral approach may be used. Alternatively, postero-lateral approaches, such as a transforaminal approach, as well as antero-lateral and oblique approaches to the spinal column have been utilized.

Accordingly, there remains a need to provide improved instruments, devices and techniques for use in accessing the disc space to insert an intervertebral implant between the vertebral bodies of adjacent vertebrae.

### SUMMARY

The disclosure is directed to a spinal implant insertion instrument and techniques for use in inserting a spinal implant in an intervertebral disc space between the vertebral bodies of adjacent vertebrae.

Accordingly, one illustrative embodiment is a spinal implant insertion instrument including a handle and an elongate shaft extending from the handle to a distal end of the elongate shaft. The insertion instrument also includes an indicator positioned proximate the handle that is pivotably coupled to the elongate shaft for providing visual indication of an angular orientation of a spinal implant removably coupled to the insertion instrument proximate the distal end of the elongate shaft.

Another illustrative embodiment is a spinal implant insertion instrument for inserting an implant in an intervertebral disc space including handle, an elongate shaft and an actuator arm which is longitudinally translatable relative to the elongate shaft. The elongate shaft extends from the handle to a distal end of the elongate shaft, and the actuator arm extends from the handle to a distal end of the actuator arm along the elongate shaft. The actuator arm includes a threaded portion proximate a proximal end of the actuator arm. The insertion instrument also includes a knob having a threaded bore for receiving the threaded portion of the actuator arm. The insertion instrument is configured such that rotation of the knob actuates the actuator arm relative to the elongate shaft to adjust an angular orientation of a spinal implant removably coupled to the insertion instrument. The insertion instrument also includes a button mounted to the handle for manipulation by a user. The button is configured to be actuated between a first position and a second position. When the button is in the first position the button is disengaged from the knob to allow rotation of the knob, and when the button is in the second position the button engages the knob to prevent rotation of the knob.

Yet another illustrative embodiment is a method of inserting a spinal implant into an intervertebral disc space between a vertebral body of a first vertebra and a vertebral body of a second vertebra of a patient. The method includes inserting the spinal implant pivotably coupled to an elongate shaft of an insertion instrument into the intervertebral disc space. The spinal implant is then pivoted relative to the elongate shaft to alter an angular orientation between a surface of the spinal implant and a longitudinal axis of the elongate shaft while the spinal implant is in the intervertebral disc space. The angular orientation of the spinal implant relative to the elongate shaft is then confirmed by observing an indicator pivotably coupled to the elongate shaft exterior of the patient.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIGS. 1-3 are perspective views of an exemplary insertion instrument for inserting a spinal implant;

FIG. 4 is a top view of the insertion instrument of FIGS. 1-3;

FIG. 5 is a first side view of the insertion instrument of FIGS. 1-3;

FIG. 6 is a second side view of the insertion instrument of FIGS. 1-3;

FIG. 7 is an exploded view of the insertion instrument of FIGS. 1-3;

FIG. 8 is a longitudinal cross-sectional view of the insertion instrument taken along line 8-8 of FIG. 4;

FIG. 8A is an enlarged view of a portion of the insertion instrument shown in FIG. 8;

FIG. 9A is a cross-sectional view of the insertion instrument taken along line 9-9 of FIG. 5 showing the button disengaged from the knob;

FIG. 9B is a cross-sectional view of the insertion instrument taken along line 9-9 of FIG. 5 showing the button engaged with the knob;

FIG. 10 illustrates actuation of a spinal implant removably coupled to the insertion instrument; and

FIGS. 11A-11C illustrate an exemplary technique for inserting a spinal implant into a disc space using the insertion tool of FIGS. 1-3.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

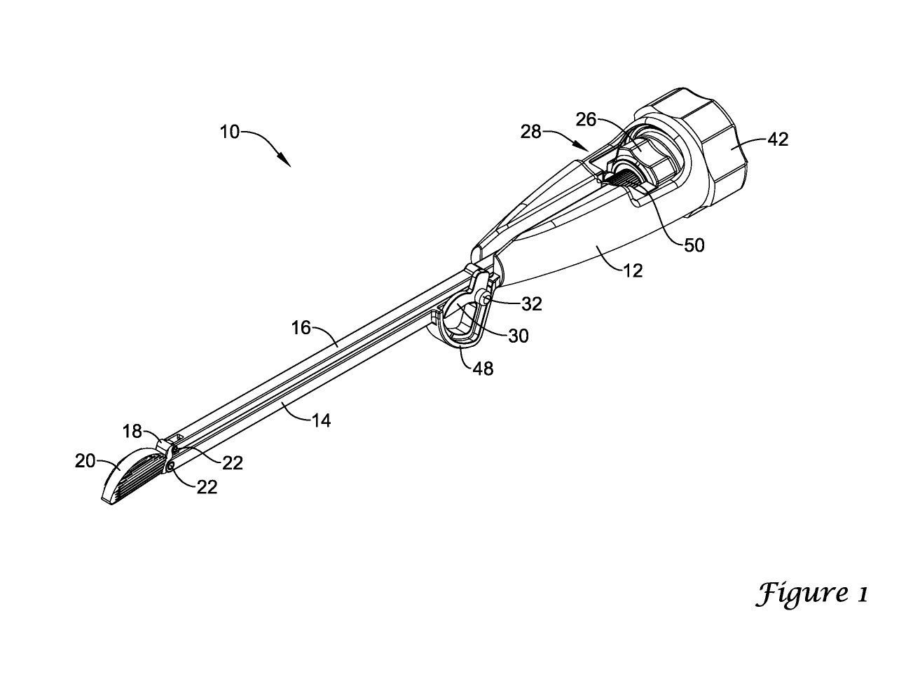

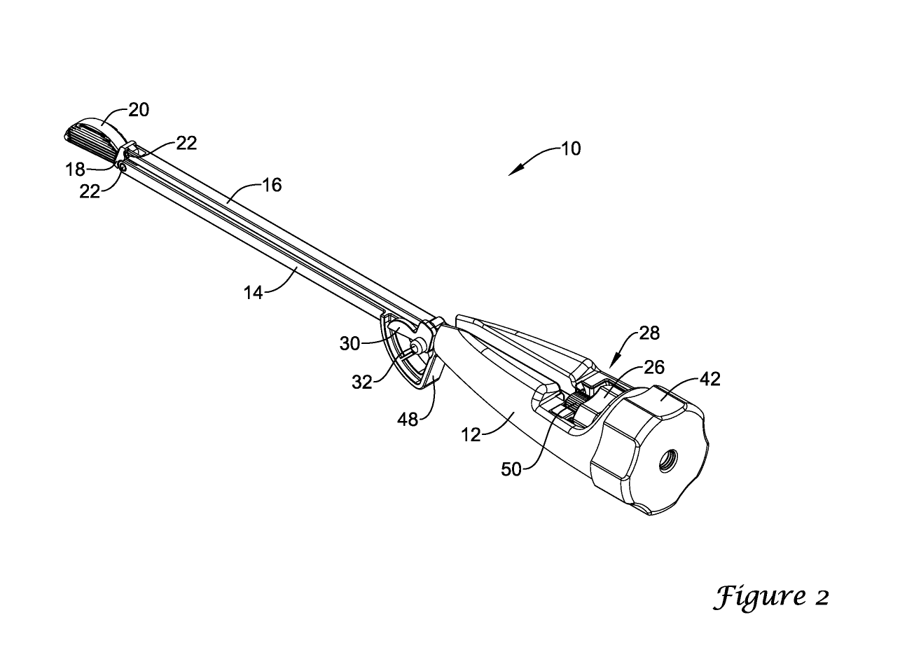

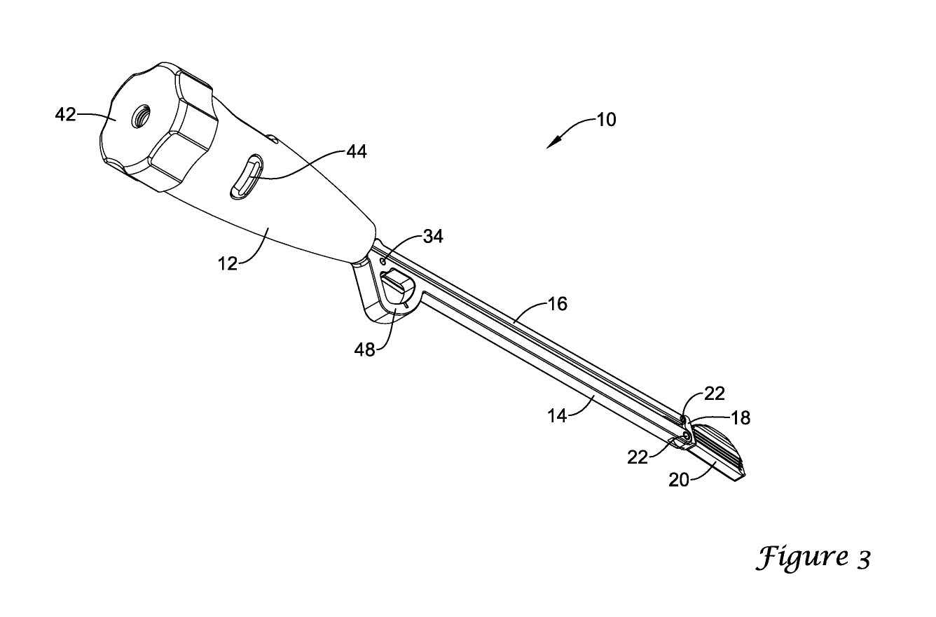

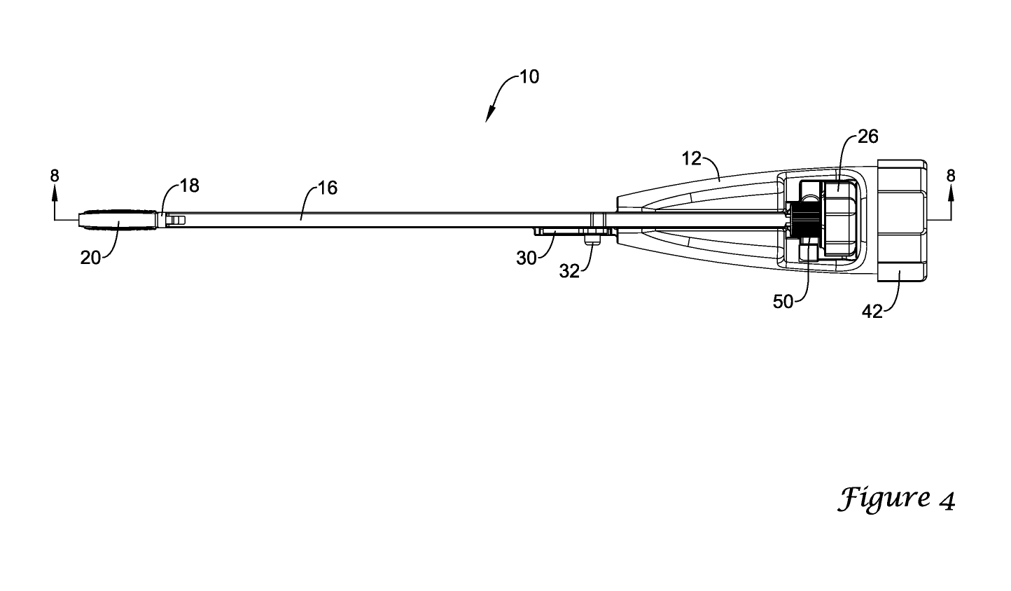

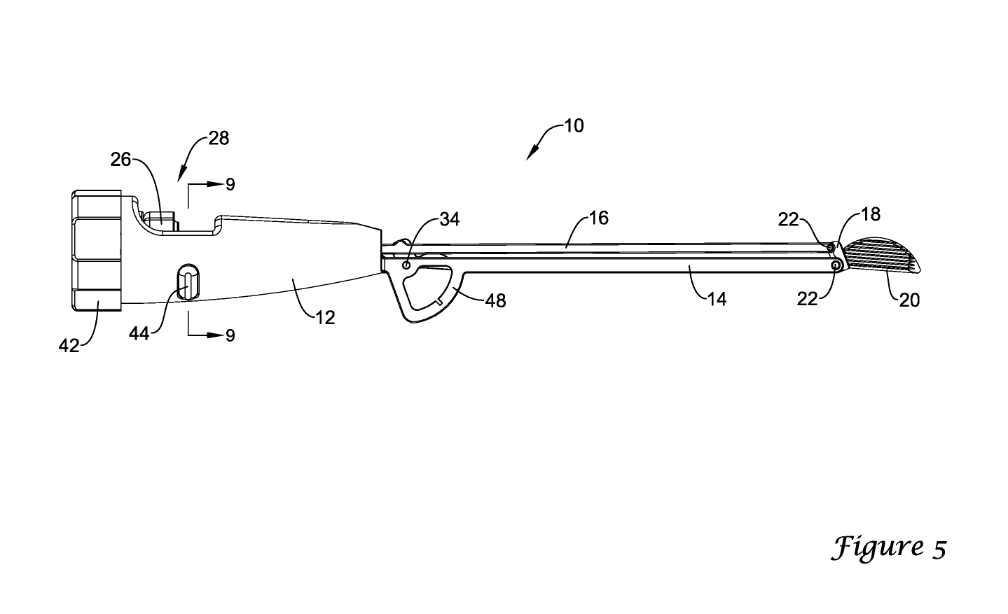

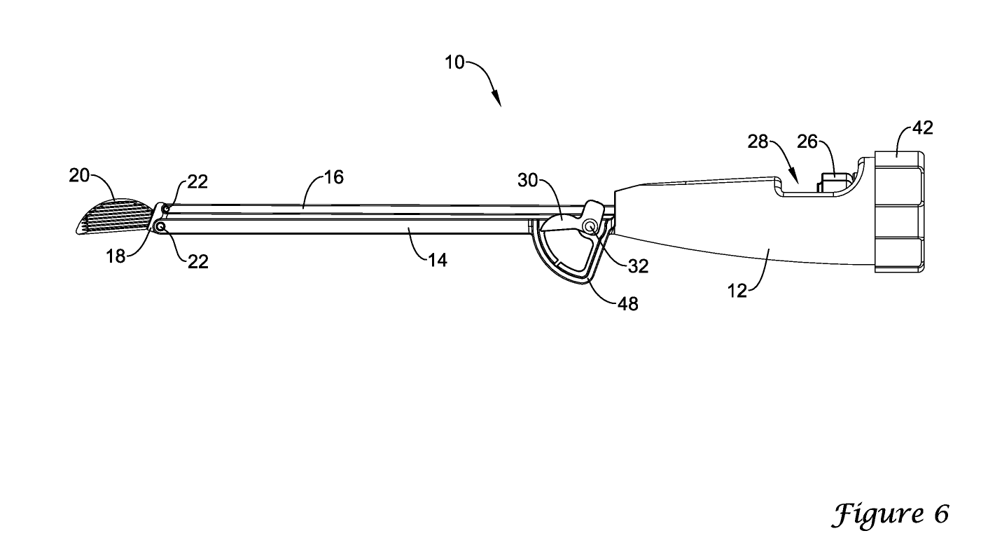

An insertion instrument 10 for inserting a spinal implant 20 into a disc space between adjacent vertebrae is shown in various orientations in FIGS. 1-6. The insertion instrument 10 may include a handle 12 and an elongate shaft 14 extending from the handle 12 to a distal end of the elongate shaft 14. In some instances, the elongate shaft 14 may be integrally formed with the handle 12 or fixedly secured to the handle 12. The insertion instrument 10 may also include an actuator arm 16 extending from the handle 12 along the elongate shaft 14. The actuator arm 16 may be longitudinally actuatable relative to the elongate shaft 14. Actuation of the actuator arm 16 relative to the elongate shaft 14 may actuate (e.g., pivot) a spinal implant 20 removably coupled to the insertion instrument 10. For example, actuation of the actuator arm 16 relative to the elongate shaft 14 may alter the angular orientation of the implant 20 relative to the longitudinal axis of the elongate shaft 14.

The insertion instrument 10 may include a tamp 18 pivotably coupled to the distal end of the elongate shaft 14 and/or pivotably coupled to the distal end of the actuator arm 16 with pins 22. The implant 20 may be removably coupled to the tamp 18 in any desired fashion, such as with complementary engagement portions. For example, the tamp 18 may include a projection which may be inserted into an opening of the implant 20 to attach the implant 20 thereto. Longitudinal translation of the actuator arm 16 relative to the elongate shaft 14 may pivot the tamp 18, and thus alter the angular orientation of the implant 20 coupled to the tamp 18.

The insertion instrument 10 may also include an indicator 30 pivotably coupled to the elongate shaft 14 for providing visual indication of the angular orientation of the implant 20 removably coupled to the insertion instrument 10 proximate the distal end of the elongate shaft 14. The indicator 30 may be positioned proximate the handle 12. Thus, during a medical procedure the indicator 30 may remain visible (i.e., in line of sight) to the medical personnel while the implant 20 is being inserted into the disc space. For instance, the indicator 30 may be located on the insertion instrument 10 such that the indicator 30 remains exterior of a patient during a medical procedure in which the insertion instrument 10 is used to insert the implant 20 into a disc space. Thus, the indicator 30 may remain exterior of an incision made in the patient's skin to access the vertebral space or may be positioned in a cavity formed by retracting tissue to access the vertebral space such that the indicator 30 is visible to the medical personnel grasping the insertion instrument 10 and inserting the implant 20.

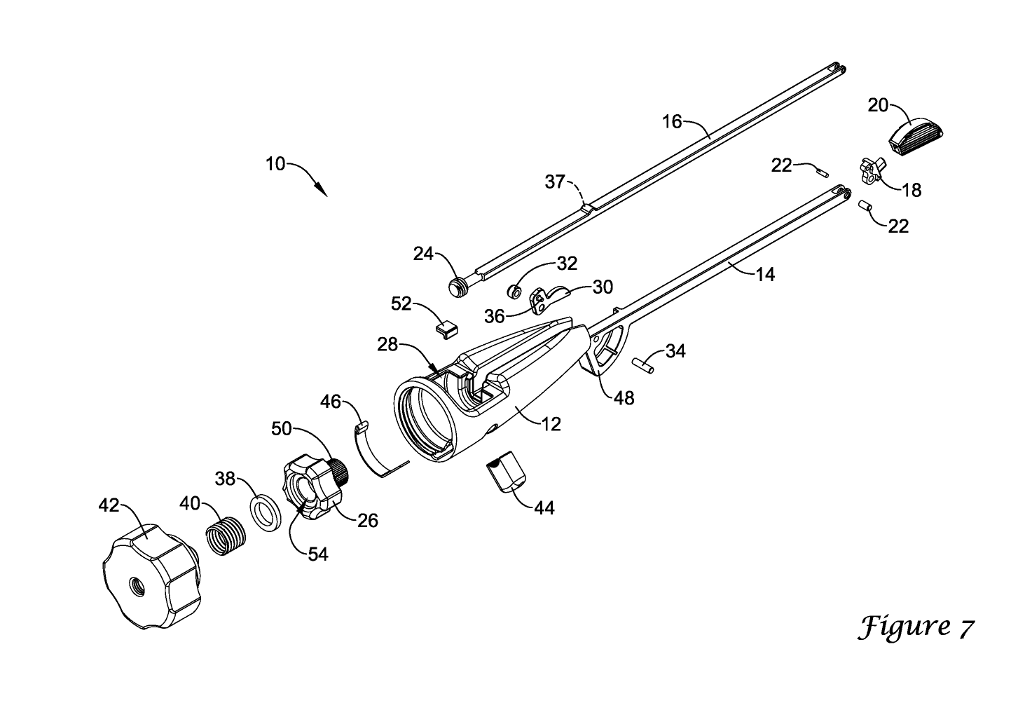

The indicator 30, which may be shaped to generally match the shape of the implant 20, may pivot simultaneously with the implant 20 to provide a visual indication to a user of the angular orientation of the implant 20 during use. In some instances, the indicator 30, while having generally the same shape of the implant 20, may be about 75% or less, 50% or less, or 25% or less than the size of the implant 20. The indicator 30 may be pivotably attached to the elongate shaft 14 via a pin 34 with the cap 32. The indicator 30 may also be pivotably attached to the actuator arm 16 such that actuation (e.g., longitudinal translation) of the actuator arm 16 causes the indicator 30 to pivot. For instance, as shown in FIG. 7, the indicator 30 may include a pin 36 which may be inserted into an opening 37 in the actuator arm 16, allowing pivotable motion of the indicator 30 relative to the actuator arm 16.

The insertion instrument 10 may be provided with a gauge 48 for gauging the angular orientation of the indicator 30, and thus determining the angular orientation of the implant 20, relative to the longitudinal axis of the elongate shaft 14. For example, the gauge 48, which may be attached to the elongate shaft 14 proximate the handle 12, may include visual indicia, such as tick marks, that may indicate the number of degrees a reference point of the indicator 30, and thus a corresponding reference point of the implant 20 is angled from the longitudinal axis of the elongate shaft 14. For example, tick marks may be arranged on the gauge 48 at desired intervals between 0° to 60°, 0° to 45°, or 0° to 30°, for example. For instance, the gauge 48 may include a tick mark at 5° intervals between 0° to 60°, between 0° to 45°, or between 0° to 30°, in some instances.

The insertion instrument 10 may also include one or more components which may be manipulated by a user to effect longitudinal translation of the actuator arm 16 along the elongate shaft 14 to pivotably actuate the implant 20. For instance, the insertion instrument 10 may include an assembly of components housed in an interior compartment 28 of the handle 12 configured to adjust the angular orientation of the implant 20 when manipulated by a user. For example, the insertion instrument 10 may include a knob 26 rotatably mounted in the compartment 28 of the handle 12.

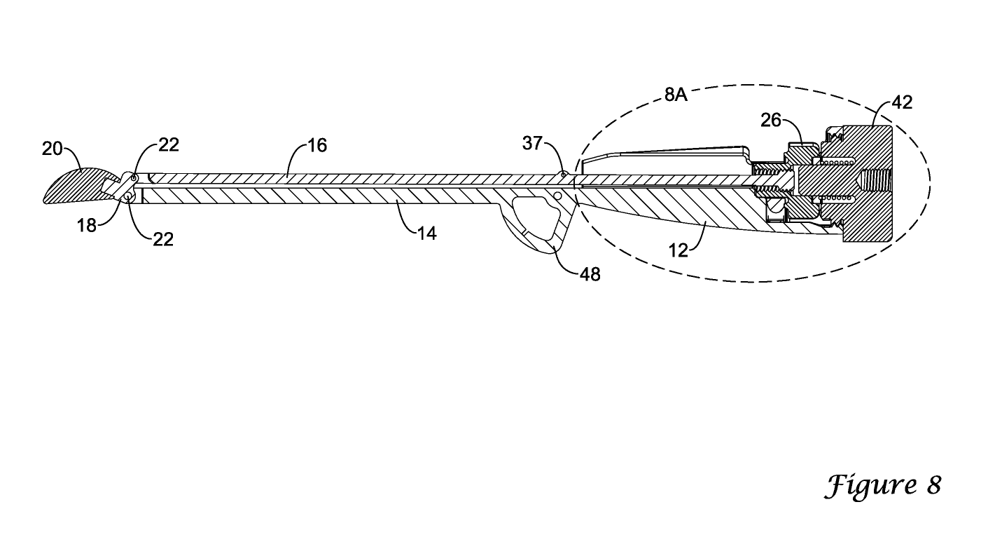

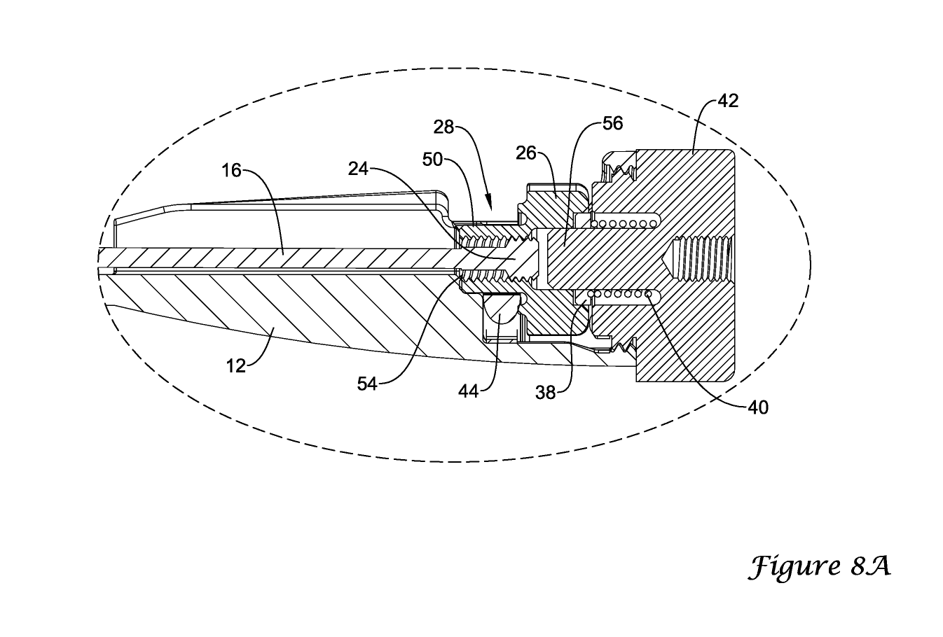

As shown in FIG. 7, the actuator arm 16 may include an externally threaded portion 24 proximate a proximal end of the actuator arm 16 that is threadably received in a threaded bore 54 of the knob 26. Thus, the knob 26 may be positioned coaxial with the actuator arm 16. As shown in FIGS. 8 and 8A, the knob 26 is arranged in the compartment 28 of the handle 12, with the threaded portion 24 of the actuator arm 16 threadably engaged with the threaded bore 54, such that rotation of the knob 26 actuates (e.g., longitudinally translates) the actuator arm 16 relative to the elongate shaft 14. Thus, rotation of the knob 26 causes the implant 20 to pivot relative to the elongate shaft 14, thus adjusting the angular orientation of the implant 20 relative to the longitudinal axis of the elongate shaft 14. Likewise, rotation of the knob 26 causes the indicator 30 to pivot relative to the elongate shaft 14, thus adjusting the angular orientation of the indicator 30 relative to the longitudinal axis of the elongate shaft 14.

As shown in FIGS. 8 and 8A, the knob 26 may be assembled in the compartment 28 such that the knob 26 may be rotated by a user. For example, a post 56 of the striking cap 42 may extend into a proximal bore of the knob 26 such that the knob 26 is coaxial with the post 56 and rotates about the axis of the post 56. The striking cap 42 may be secured to the proximal end of the handle 12, such as by threaded portions shown in FIGS. 8 and 8A, or other securement means. The assembly may include a coil spring 40 positioned in a cavity of the striking cap 42 around the post 56 configured to apply pressure against the knob 26 through a bearing 38 positioned between the coil spring 40 and a proximal annular rim of the knob 26. Frictional forces applied to the knob 26 by the coil spring 40 may help retain the knob 26 in position and provide some degree of resistance to rotation of the knob 26 to preclude inadvertent rotation of the knob 26.

The insertion instrument 10 may also include a mechanism which is actuatable by a user for selectively preventing and selectively permitting rotation of the knob 26, and thus actuation of the actuator arm 16. For example, the insertion instrument 10 may include a button 44 or other mechanism (e.g., lever, switch, dial, screw, etc.) on the handle 12 configured for manipulation by a user to selectively engage the knob 26 to prevent rotation of the knob 26. The button 44 may extend through an opening in the handle 12 from exterior of the handle 12 to the knob 26. Thus a user may manipulate the button 44 as a user grasps the handle 12 with a hand of the user.

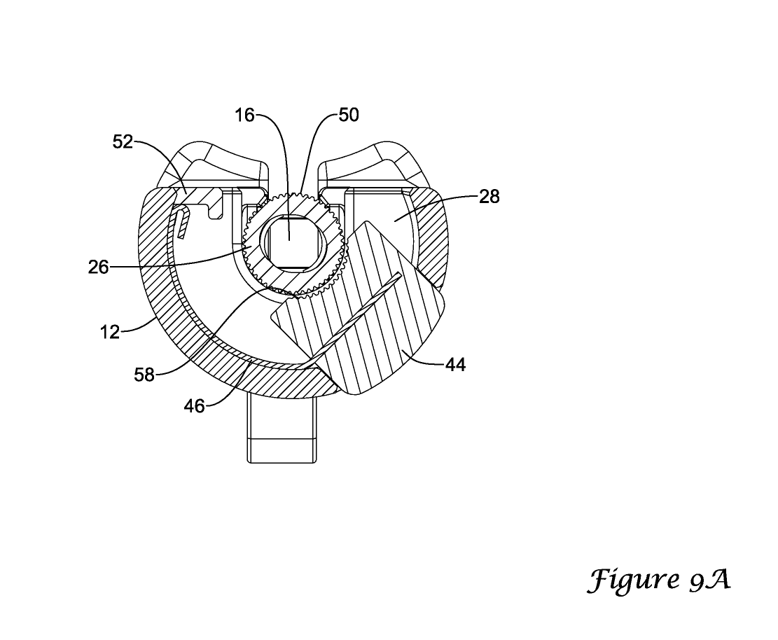

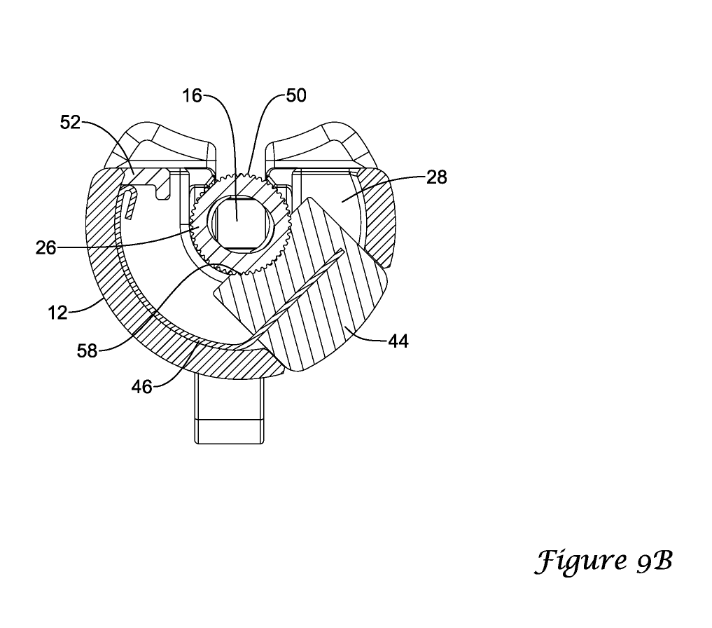

The button 44 may be actuatable between a first position, shown in FIG. 9A, and a second position, shown in FIG. 9B. When the button 44 is in the first position, the button 44 is disengaged from the knob 26 to allow rotation of the knob 26. When the button 44 is in the second position, the button 44 engages the knob 26 to prevent rotation of the knob 26. In some instances, the button 44 may be biased to the first position such that the button 44 is normally disengaged from the knob 26 to allow rotation of the knob 26. However, in other instances, the button 44 may be biased to the second position such that the button 44 is normally engaged with the knob 26 to prevent rotation of the knob 26. The button 44 may be biased away from the knob 26 with a resilient member such as a spring 46 for biasing the button 44 to the first position. The spring 46 may have any desired configuration. As shown in FIGS. 9A and 9B, in some instances, the spring 46 may be generally L-shaped, with a curved portion resting against a curved surface of the compartment 28 in the handle 12, while a projection portion of the spring 46 is positioned into the button 44 and secured thereto. The stopper 52, which may be an integral portion of the handle 12 or a separate component, may retain the spring 46 in position in the compartment 28, such as in a groove formed in the compartment 28 for receiving the spring 46.

The knob 26 may include a protruding boss 50 against which the button 44 is selectively engaged. The boss 50 may include one or more engagement features configured to engage with one or more engagement features of the button 44, or other mechanism, when the button 44, or other mechanism, is actuated to the second position. For example, the boss 50 may include a plurality of splines or flutes on a peripheral outer surface thereof configured to mate with a surface of the button 44, such as a grooved surface 58 of the button 44. In some instances, the grooved surface 58 may be concave to mate with the outer surface of the boss 50 over a larger arc length. It is noted that other engagement features may be provided to provide selective engagement and disengagement of the button 44 with the knob 26.

During use, the user may press the button 44, and thus actuate the button 44 to the second, engaged position as a user grasps the handle 12 with one hand of the user. While grasping the handle 12 with a first hand, which also engages the button 44 with the knob 26, the user may strike the striking cap 42 of the insertion instrument 10 with a striking tool (e.g. a hammer) held in a second hand to drive the implant 20 into the intervertebral disc space between adjacent vertebrae. With the button 44 engaged with the knob 26, striking the insertion instrument 10 will not cause inadvertent rotation of the knob 26 and/or inadvertent change in the angular orientation of the implant 20 during impaction. Striking the striking cap 42 with a striking force may transfer the striking force through the insertion instrument 10 to the implant 20 to forcibly insert the implant 20 into proper position in the disc space between adjacent vertebrae.

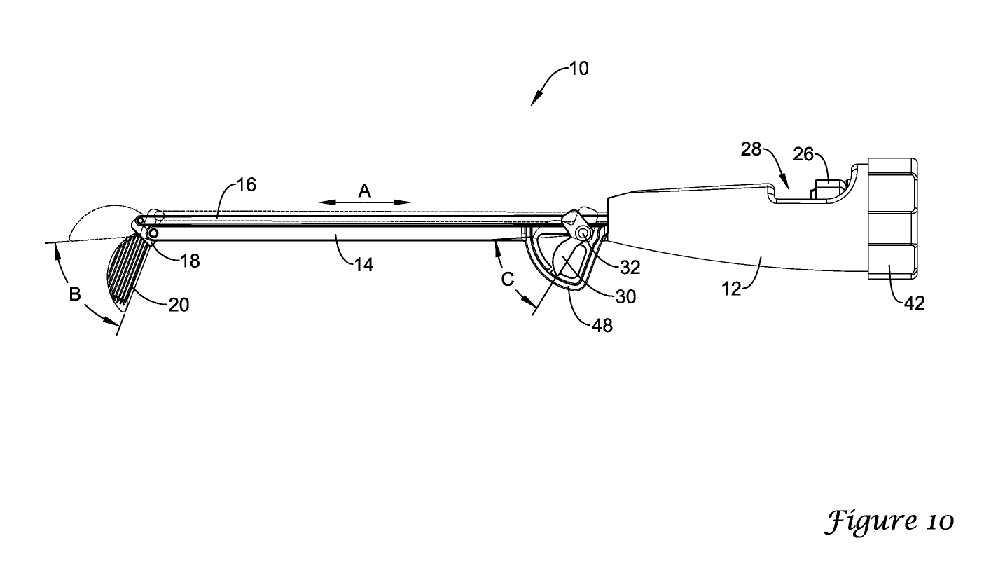

Referring now to FIG. 10, angular adjustment of the implant 20 relative to the longitudinal axis of the elongate shaft 14 of the insertion instrument 10 is illustrated in which the pivotal position of the implant 20 can be altered between a first position (shown in dashed lines) and a second position (shown in solid lines) through actuation of the actuator arm 16 via rotation of the knob 26. For example, longitudinal translation of the actuator arm 16 along path or range of motion A may cause the implant 20 to pivot along an arcuate path or range of motion B. Correspondingly, the indicator 30 may also pivot along an arcuate path or range of motion C, representative of the arcuate path or range of motion B which the implant 20 travels through.

The indicator 30 may be configured such that the angular orientation of the indicator 30 relative to the longitudinal axis of the elongate shaft 14 is representative of the angular orientation of the implant 20 relative to the longitudinal axis of the elongate shaft 14. For example, the indicator 30 may be configured such that the angular orientation of a reference surface of the indicator 30 relative to the longitudinal axis of the elongate shaft 14 is equal to (or otherwise is indicative of) the angular orientation of a reference surface of the implant 20 relative to the longitudinal axis of the elongate shaft 14.

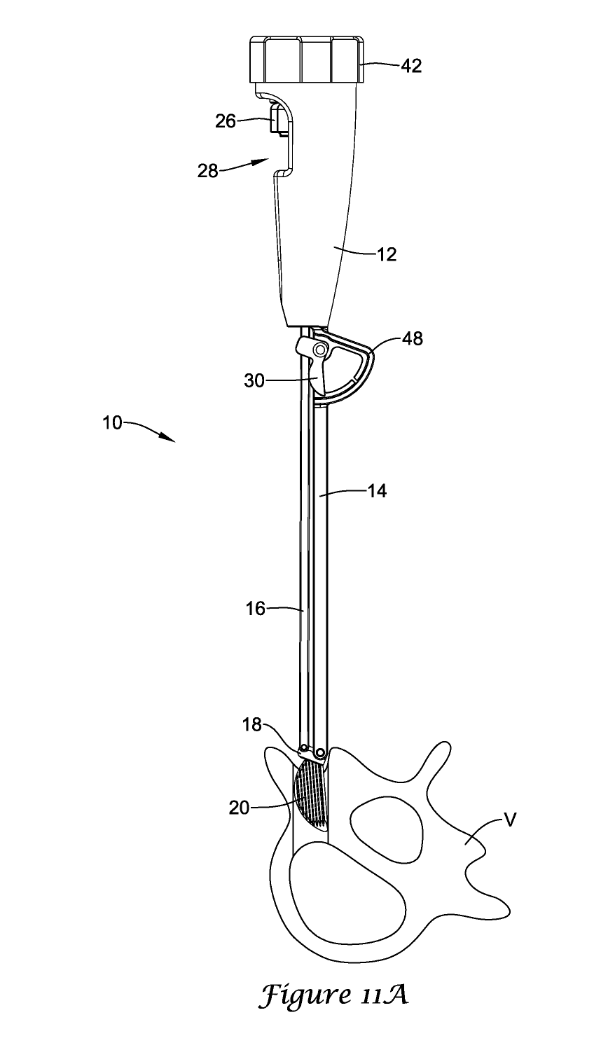

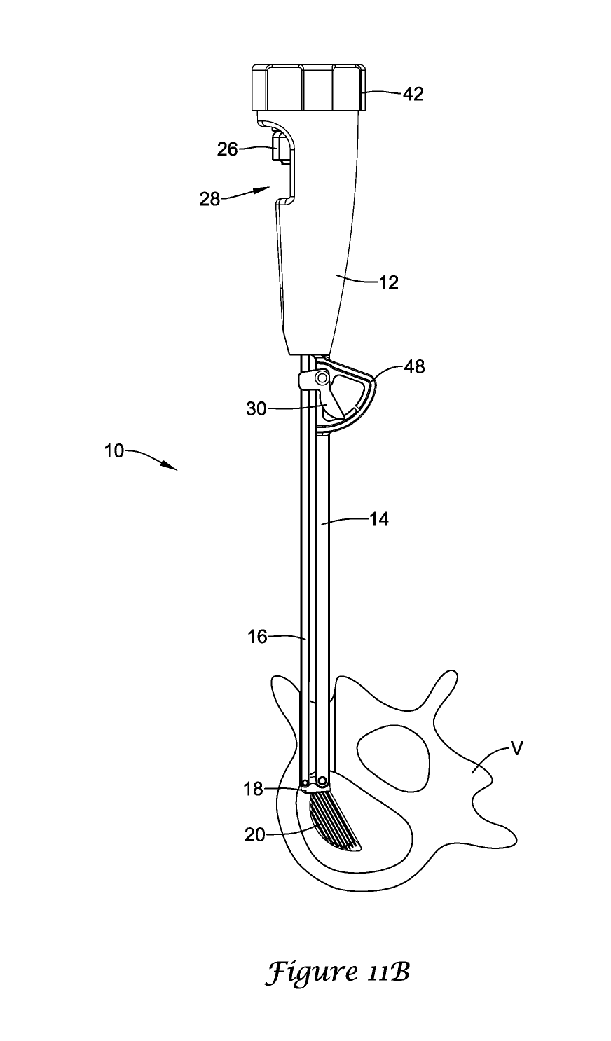

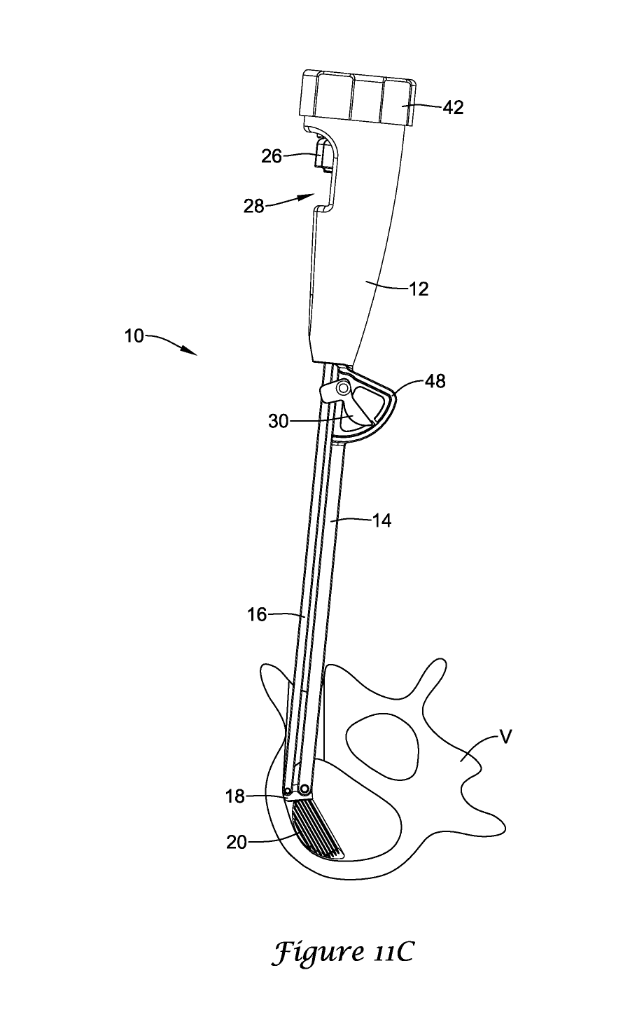

FIGS. 11A-11C illustrate an exemplary method of inserting the implant 20 into the intervertebral disc space between the vertebral bodies of adjacent vertebrae V. Although the described method uses a postero-lateral approach, other approaches such as anterior, posterior, lateral, antero-lateral and oblique approaches to the spinal column may be utilized if desired.

As shown in FIG. 11A, the implant 20 may initially be substantially in line with the longitudinal axis of the elongate shaft 14 of the insertion instrument 10 as the implant 20 is first inserted into the patient's body to the intervertebral disc space between adjacent vertebrae V of the patient's spinal column. In some instances, the surgeon may apply a striking force to the insertion instrument 10 to drive the implant 20 into the disc space with the implant 20 substantially in line with the longitudinal axis of the elongate shaft 14. While applying a striking force, the surgeon may press the button 44 on the handle 12 of the insertion instrument 10 while grasping the handle 12 with one hand in order to engage the button 44 against the splines of the boss 50 of the knob 26 to prevent pivotable movement of the implant 20 relative to the elongate shaft 14.

Once the implant 20 is positioned in or proximate the disc space, the surgeon may then release the button 44, if not already released, and pivot the implant 20 relative to the elongate shaft 14 to an alternative orientation in which the implant 20 is at an angular orientation to the elongate shaft 14, shown in FIG. 11B, by manually rotating the knob 26. The surgeon may confirm the angular orientation of the implant 20 relative to the elongate shaft 14 with the implant 20 in the disc space by visually observing the indicator 30 pivotably coupled to the elongate shaft 14 which remains exterior of the patient or otherwise in line of sight of the surgeon. With the implant 20 at a desired angular orientation, the surgeon may press the button 44 on the handle 12 of the insertion instrument 10 while grasping the handle 12 with one hand in order to engage the button 44 against the splines of the boss 50 of the knob 26 to lock the knob 26 from further rotation in order to prevent further pivotable movement of the implant 20 relative to the elongate shaft 14. While pressing the button 44, the surgeon may strike the insertion instrument 10 with a striking tool to drive the implant 20 further into the disc space.

The surgeon may then release the button 44 and pivot the implant 20 relative to the elongate shaft 14 to another alternative orientation in which the implant 20 is typically at an even greater angular orientation to the elongate shaft 14, shown in FIG. 11C, by manually rotating the knob 26. The surgeon may confirm the angular orientation of the implant 20 relative to the elongate shaft 14 with the implant 20 in the disc space by visually observing the indicator 30 pivotably coupled to the elongate shaft 14 which remains exterior of the patient or otherwise in line of sight of the surgeon. With the implant 20 at a desired angular orientation, the surgeon may again press the button 44 on the handle 12 of the insertion instrument 10 while grasping the handle 12 with one hand in order to engage the button 44 against the splines of the boss 50 of the knob 26 to lock the knob 26 from further rotation in order to prevent further pivotable movement of the implant 20 relative to the elongate shaft 14. While pressing the button 44, the surgeon may again strike the insertion instrument 10 with a striking tool to further drive the implant 20 further into the disc space.

This process of altering the angular orientation of the implant 20 by rotating the knob 26 to actuate the actuator arm 16, followed by pressing the button 44 to prevent further rotation and applying a striking force to the insertion instrument 10 to impact the implant 20 into the disc space may be repeated until the implant 20 is properly positioned in the disc space. The insertion instrument 10 may then be uncoupled from the implant 20 and removed from the patient's body. In circumstances in which another implant 20 is desired on a contra-lateral side of the disc space, the technique may be repeated with another implant 20.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A spinal implant insertion instrument comprising:
a handle;
an elongate shaft extending from the handle to a distal end of the elongate shaft; and
an indicator pivotably coupled to the elongate shaft for providing visual indication of an angular orientation of a spinal implant removably coupled to the insertion instrument proximate the distal end of the elongate shaft.

2. The spinal implant insertion instrument of claim 1, wherein the indicator is configured such that an angular orientation the indicator relative to a longitudinal axis of the elongate shaft is representative of an angular orientation the spinal implant relative to the longitudinal axis of the elongate shaft.

3. The spinal implant insertion instrument of claim 1, wherein the indicator is configured such that an angular orientation of a surface of the indicator relative to a longitudinal axis of the elongate shaft is equal to an angular orientation of a surface of the spinal implant relative to the longitudinal axis of the elongate shaft.

4. The spinal implant insertion instrument of claim 1, wherein the indicator pivots simultaneously with the spinal implant.

5. The spinal implant insertion instrument of claim 1, wherein the indicator is shaped to generally match a shape of the spinal implant.

6. The spinal implant insertion instrument of claim 1, wherein the indicator is positioned proximate the handle such that the indicator remains exterior of a patient during a medical procedure.

7. The spinal implant insertion instrument of claim 1, further comprising an actuator arm longitudinally actuatable relative to the elongate shaft, wherein the indicator is pivotably coupled to each of the actuator arm and the elongate shaft.

8. The spinal implant insertion instrument of claim 7, further comprising a knob which is rotatable to actuate the actuator arm relative to the elongate shaft.

9. The spinal implant insertion instrument of claim 8, further comprising a button mounted to the handle which is selectively actuatable to prevent rotation of the knob.

10. A spinal implant insertion instrument for inserting an implant in an intervertebral disc space, the insertion instrument comprising:
a handle;
an elongate shaft extending from the handle to a distal end of the elongate shaft;
an actuator arm extending from the handle to a distal end of the actuator arm, the actuator arm including a threaded portion proximate a proximal end of the actuator arm;
a knob having a threaded bore for receiving the threaded portion of the actuator arm, wherein rotation of the knob actuates the actuator arm relative to the elongate shaft to adjust an angular orientation of a spinal implant removably coupled to the insertion instrument; and
a button mounted to the handle for manipulation by a user, the button configured to be actuated between a first position and a second position, wherein when the button is in the first position the button is disengaged from the knob to allow rotation of the knob and when the button is in the second position the button engages the knob to prevent rotation of the knob.

11. The spinal implant insertion instrument of claim 10, further comprising an indicator pivotably coupled to the elongate shaft for providing visual indication of an angular orientation of a spinal implant removably coupled to the insertion instrument.

12. The spinal implant insertion instrument of claim 11, wherein actuation of the actuator arm relative to the elongate shaft through rotation of the knob causes the indicator to pivot relative to the elongate shaft.

13. The spinal implant insertion instrument of claim 10, wherein the button is biased to the first position.

14. The spinal implant insertion instrument of claim 13, further comprising a spring coupled to the button for biasing the button to the first position.

15. The spinal implant insertion instrument of claim 13, wherein the button is configured to be actuated to the second position as a user grasps the handle with a hand of the user.

16. The spinal implant insertion instrument of claim 10, wherein the button includes a grooved surface configured to engage a plurality of splines on the knob.

17. The spinal implant insertion instrument of claim 16, wherein the grooved surface of the button is concave.

18. A method of inserting a spinal implant into an intervertebral disc space between a vertebral body of a first vertebra and a vertebral body of a second vertebra of a patient, the method comprising:
inserting the spinal implant pivotably coupled to an elongate shaft of an insertion instrument into the intervertebral disc space;
pivoting the spinal implant relative to the elongate shaft to alter an angular orientation between a surface of the spinal implant and a longitudinal axis of the elongate shaft while the spinal implant is in the intervertebral disc space; and
confirming the angular orientation of the spinal implant relative to the elongate shaft by observing an indicator pivotably coupled to the elongate shaft exterior of the patient.

19. The method of claim 18, further comprising:
pressing a button on a handle of the insertion instrument while grasping the handle of the insertion instrument in order to prevent further pivotable movement of the spinal implant relative to the elongate shaft; and
striking the insertion instrument with a striking tool to drive the spinal implant further into the interververtebral disc space.

20. The method of claim 19, wherein the insertion instrument includes a knob which is rotatable to pivot the spinal implant relative to the elongate shaft, wherein when the button is pressed the button engages the knob to prevent rotation of the knob.
